(19) United States
(12) Patent Application Publication
(10) Pub. No.: US 2007/0276369 A1
(43) Pub. Date: Nov. 29, 2007

(54) In vivo-customizable implant

(75) Inventors: Randall N. Allard, Germantown, TN (US); Kent M. Anderson, Memphis, TN (US); Eric C. Lange, Collierville, TN (US); Aurelien Bruneau, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/442,621

(22) Filed: May 26, 2006

(51) Int. Cl. A61F 2/30

(52) U.S. Cl. 606/61

(57) ABSTRACT

A spinal implant, implant control device and method of treating a spine are provided. An exemplary spinal implant can include an adjustable component and a connector in communication with the adjustable component, wherein the connector is configured for transcutaneous delivery of an agent to the adjustable component in a manner that affects a condition of the adjustable component.

### FIELD OF THE DISCLOSURE

The present disclosure relates generally to systems and methods for regulating and/or customizing implants in vivo. More specifically, the present disclosure relates to postoperative adjustment and/or regulation of surgical implants.

### BACKGROUND

In human anatomy, the spine is a generally flexible column that can take tensile and compressive loads. The spine also allows bending motion and provides a place of attachment for keels, muscles and ligaments. Generally, the spine is divided into four sections: the cervical spine, the thoracic or dorsal spine, the lumbar spine, and the pelvic spine. The pelvic spine generally includes the sacrum and the coccyx. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column can be subjected. At the same time, the intervertebral discs can allow adjacent vertebral bodies to move relative to each other, particularly during bending or flexure of the spine. Thus, the intervertebral discs are under constant muscular and gravitational pressure and generally, the intervertebral discs are the first parts of the lumbar spine to show signs of deterioration.

In particular, deterioration can be manifested as a herniated disc. Weakness in an annulus fibrosis can result in a bulging of the nucleus pulposus or a herniation of the nucleus pulposus through the annulus fibrosis. Ultimately, weakness of the annulus fibrosis can result in a tear permitting the nucleus pulposus to leak from the intervertebral space. Loss of the nucleus pulposus or a bulging of the nucleus pulposus can lead to a reduction in the intervertebral space resulting in pinching of nerves and contact between osteal surfaces. This condition can cause pain and damage to vertebrae. In addition, aging can lead to a reduction in the hydration of the nucleus pulposus. Such a loss in hydration can also permit contact between osteal surfaces and pinching of nerves.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

### BRIEF DESCRIPTION OF THE DRAWINGS

The present disclosure may be better understood, and its numerous features and advantages made apparent to those skilled in the art by referencing the accompanying drawings, wherein:

FIG. 1 includes a lateral view of a portion of a vertebral column;

FIG. 2 includes a lateral view of a pair of adjacent vertebrae;

FIG. 3 includes a top plan view of a vertebra;

FIG. 4 includes a cross sectional view of an intervertebral disc;

FIG. 5 includes a plan view of an interspinous process brace in a deflated configuration;

FIG. 6 includes a plan view of an interspinous process brace in an expanded configuration;

FIG. 7 includes a plan view of an interspinous process brace in an expanded configuration with a tether installed there around;

FIG. 8 includes an anterior view of an intervertebral prosthetic disc;

FIG. 9 includes an exploded anterior view of an intervertebral prosthetic disc;

FIG. 10 includes a lateral view of an intervertebral prosthetic disc;

FIG. 11 includes an exploded lateral view of an intervertebral prosthetic disc;

FIG. 12 includes a plan view of a superior half of an intervertebral prosthetic disc;

FIG. 13 includes a plan view of an inferior half of an intervertebral prosthetic disc; and

FIG. 14 includes a diagram of a controlled release device;

The use of the same reference symbols in different drawings indicates similar or identical items.

### DESCRIPTION OF EMBODIMENTS

In an exemplary embodiment, a spinal implant can include an adjustable component and a connector in communication with the adjustable component, wherein the connector is configured for transcutaneous delivery of an agent to the adjustable component in a manner that affects a condition of the adjustable component.

In another exemplary embodiment, a spinal implant can include an adjustable component having a sealable surface configured to allow percutaneous delivery of an agent to the adjustable component in a manner that affects a condition of the adjustable component.

In another exemplary embodiment, a method of treating a spine of a patient can include the steps of determining a post surgical performance condition associated with a previously installed spinal implant and selectively releasing an agent to affect the performance condition.

In another exemplary embodiment, an implant control device can include a sensor configured to determine a performance condition associated with a spinal implant; a reservoir configured to include a first agent capable of affecting the performance condition associated with the spinal implant; a control element configured to provide access to the reservoir; and a controller in communication with the sensor and the control element. The controller can be configured to manipulate the control element to provide access to the reservoir in response to the condition determined by the sensor.

In a further exemplary embodiment, an implant control device can include a sensor configured to determine a condition associated with a spinal implant; a first reservoir configured to include a first agent; a second reservoir configured to include a second agent; and a controller in communication with the sensor. The controller can be configured to selectively initiate access to the first reservoir or the second reservoir in response to the condition determined by the sensor.

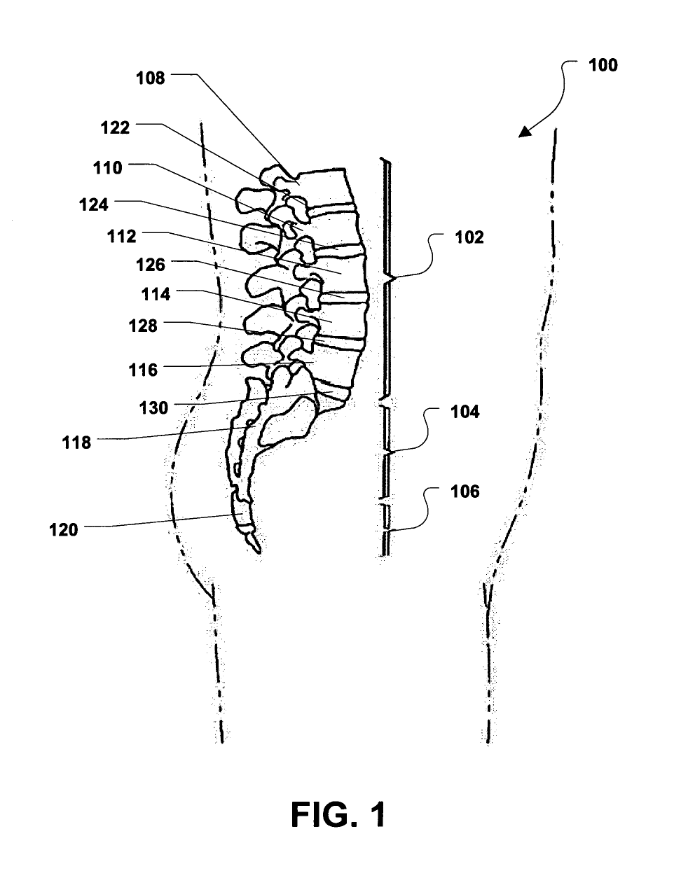

Referring initially to FIG. 1, a portion of a vertebral column, designated 100, is shown. As depicted, the vertebral column 100 includes a lumbar region 102, a sacral region 104, and a coccygeal region 106. The vertebral column 100 also includes a cervical region and a thoracic region. For clarity and ease of discussion, the cervical region and the thoracic region are not illustrated.

As illustrated in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, or damaged that intervertebral lumbar disc 122, 124, 126, 128, 130 can be at least partially treated with an implanted device and/or method according to one or more of the embodiments described herein. In a particular embodiment, a customizable spinal implant can be inserted into an intervertebral space following a discectomy. Although the general type (prosthetic disc, interprocess brace, etc.) and configuration of the spinal implant can be determined by a skilled practitioner based on clinical need and diagnostic techniques, fine adjustment of the implant based on irregularities presenting postoperatively at the implant site as well as postoperative performance issues may be accomplished according to the embodiments described herein.

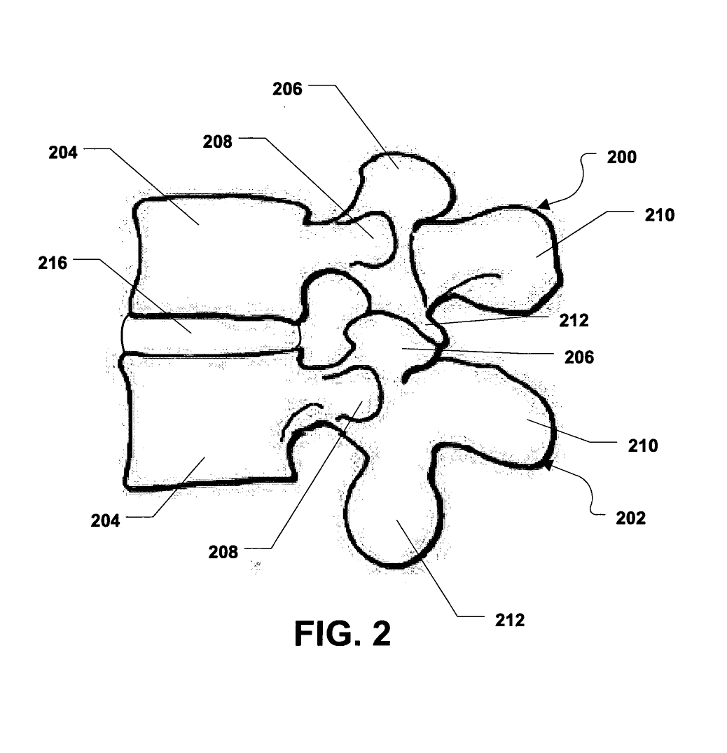

FIG. 2 depicts a detailed lateral view of two adjacent vertebrae, e.g., two of the lumbar vertebra 108, 110, 112, 114, 116 illustrated in FIG. 1. FIG. 2 illustrates a superior vertebra 200 and an inferior vertebra 202. As illustrated, each vertebra 200, 202 includes a vertebral body 204, a superior articular process 206, a transverse process 208, a spinous process 210 and an inferior articular process 212. FIG. 2 further depicts an intervertebral disc 214 between the superior vertebra 200 and the inferior vertebra 202. As described in greater detail below, a customizable interspinous process implant according to one or more of the embodiments described herein can be installed between the spinous processes 210 of adjacent vertebrae.

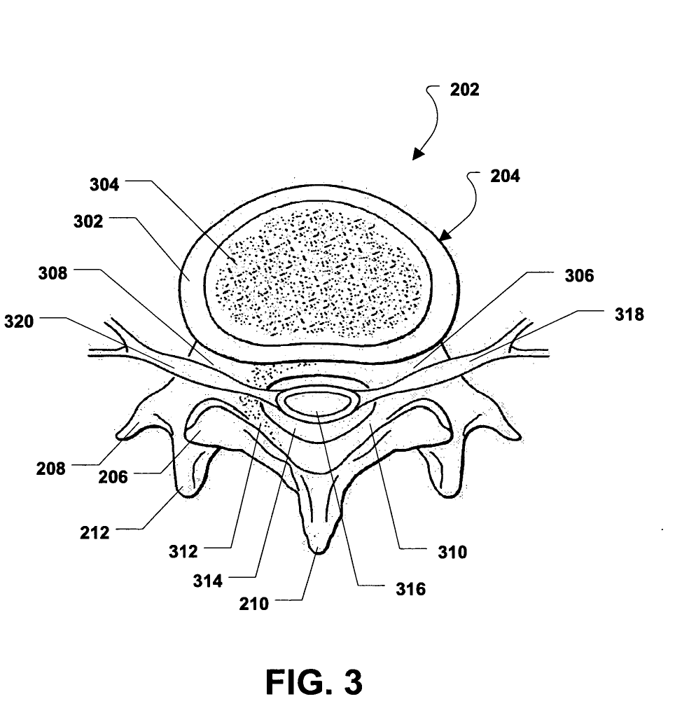

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

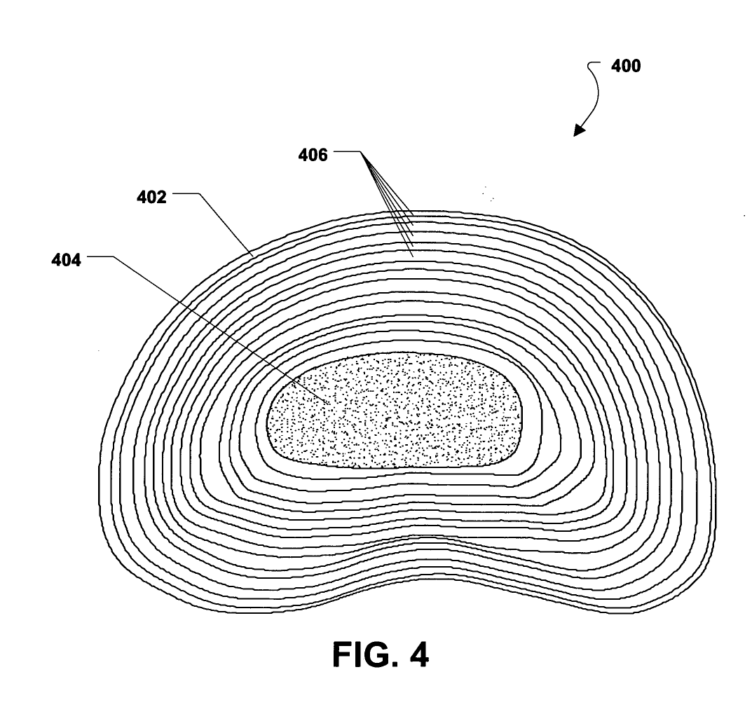

Referring now to FIG. 4, an intervertebral disc is shown and is generally designated 400. The intervertebral disc 400 is made up of two components: the annulus fibrosis 402 and the nucleus pulposus 404. The annulus fibrosis 402 is the outer portion of the intervertebral disc 400, and the annulus fibrosis 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosis 402 is a structure that is exceptionally strong, yet extremely flexible.

The nucleus pulposus 404 is the inner gel material that is surrounded by the annulus fibrosis 402. It makes up about forty percent (40%) of the intervertebral disc 400 by weight. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) by weight at birth and decreases to about seventy percent by weight (70%) by the fifth decade.

Injury or aging of the annulus fibrosis 402 can allow the nucleus pulposus 404 to be squeezed through the annulus fibers either partially, causing the disc to bulge, or completely, allowing the disc material to escape the intervertebral disc 400. The bulging disc or nucleus material can compress the nerves or spinal cord, causing pain. Accordingly, the nucleus pulposus 404 can be treated with a customizable spinal implant to improve the condition and/or performance of the intervertebral disc 400.

One aspect of the present disclosure is directed to a spinal implant that is adjustable or configurable during postoperative care. Such adjustment or configuration can include, for example, fine adjustment of the implant based on irregularities presenting postoperatively at the implant site as well as postoperative performance issues—over-extensive range of motion at the implant site, contact or compression of a nerve root, etc. Several of these types of issues may not present until postoperative care has begun and, in certain circumstances, certain issues may not present until swelling subsides or until the patient is able to move about in an upright position for extended periods or until the patient is generally active again.

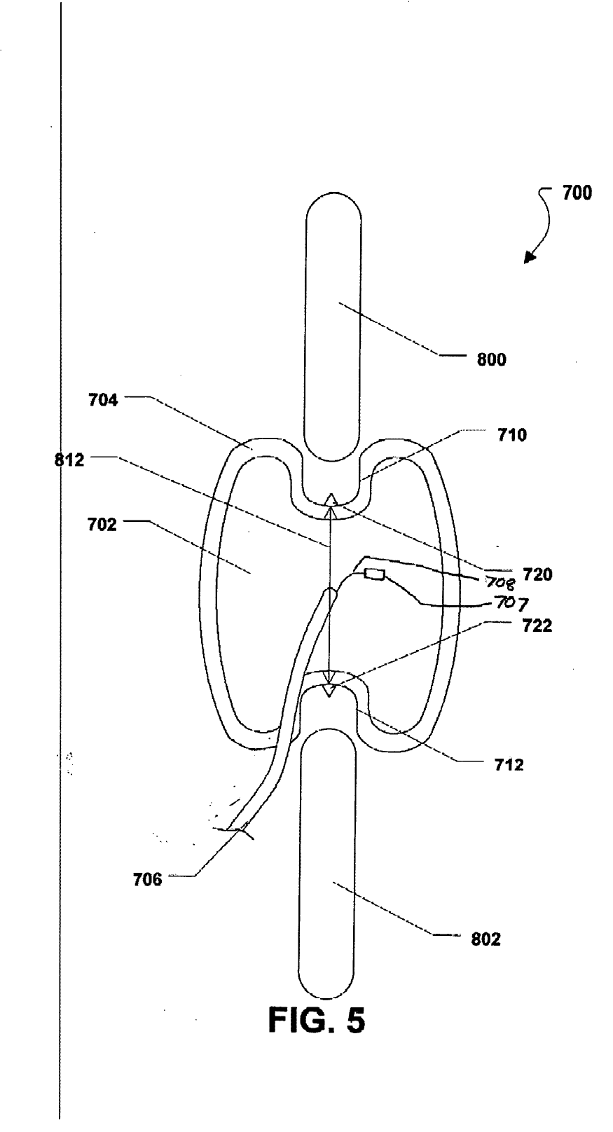

As shown in FIGS. 5-7, an exemplary embodiment of the present spinal implant is directed to an interspinous process brace identified generally as 700. As shown, the interspinous process brace 700 can include an adjustable component 702, which in this embodiment is an expandable interior chamber. The adjustable component 702 can be provided in a shape that can generally engage and/or stabilize at least one spinous process, such as, for example, the spinous processes of two adjacent vertebrae. In a particular embodiment, the adjustable component 702 can be generally H-shaped.

Further, in a particular embodiment, the adjustable component 702 can be made from one or more expandable biocompatible materials. For example, the materials can be silicones, polyurethanes, polycarbonate urethanes, polyethylene terephthalate, silicone copolymers, polyolefins, or any combination thereof. Also, the adjustable component 702 can be non-porous or micro-porous. The adjustable component can be selectively permeable. In certain embodiments in which the adjustable component contains a swellable and/or bioresorbable polymer material, the adjustable component can be formed of a selectively permeable or micro-porous material that allows fluids to flow in and/or out of the adjustable component so that hydration can be adjusted within the adjustable component in vivo.

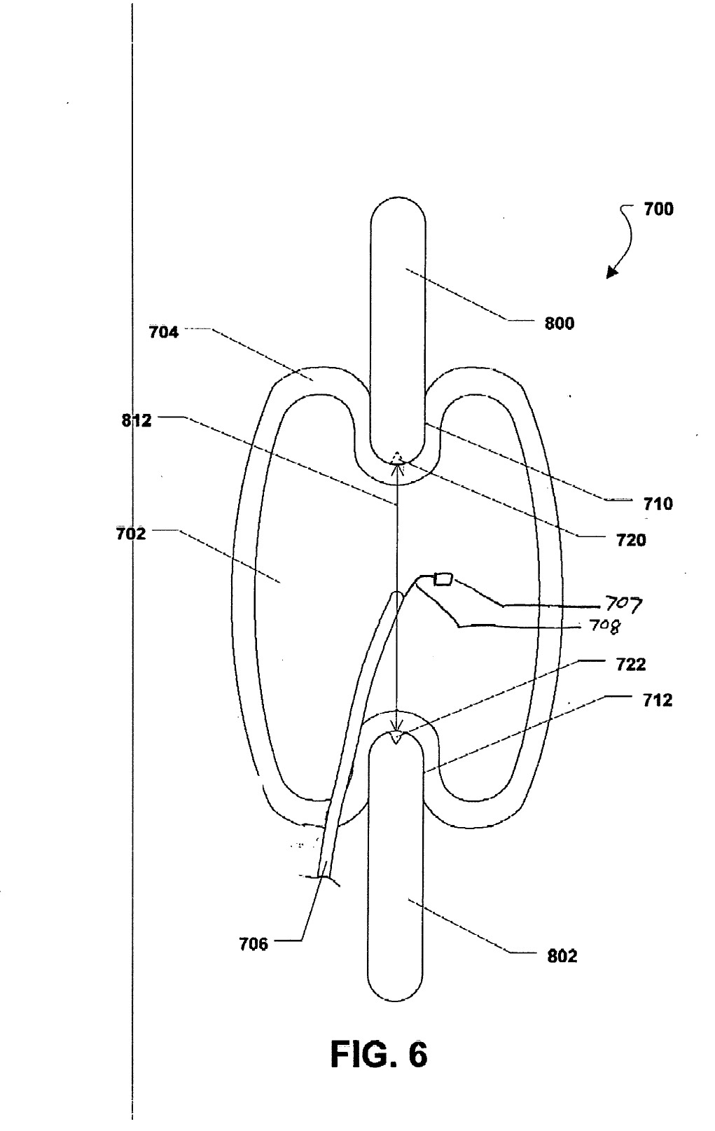

As shown in FIG. 5, the adjustable component 702 can include a connector 706. The connector 706 can be used to initially provide an injectable biocompatible material to the adjustable component 702 during installation. In a particular embodiment, the adjustable component can be expanded from a deflated configuration, shown in FIG. 5, to one of a plurality of inflated configurations, shown in FIG. 6, up to a maximum inflated configuration. Further, after the adjustable component 702 is initially inflated, or otherwise expanded, the connector 706 can be positioned transcutaneously or attached to a transcutaneous, self-sealable port in order to allow unobstructed, postoperative access to the adjustable component from outside the patient. Alternatively, the connector can include an implantable self-sealing port to allow percutaneous access to the connector.

In a particular embodiment, the expandable interspinous process brace 700 can include a one-way self-sealing valve (not shown) within the adjustable component 702 or within the connector 706. The self-sealing valve can prevent the adjustable component from leaking and thus allow pressure to be maintained against the spinous processes.

In another exemplary embodiment, a spinal implant can include an adjustable component having a sealable surface configured to allow percutaneous delivery of an agent directly to the adjustable component, i.e., without passing through a connector. The sealable surface can be a portion of a side of the implant (e.g., a window), such as a portion of the posterior side. In other embodiments, the sealable surface can comprise the entire side or multiple sides of the implant such that the agent can be delivered percutaneously through a needle with or without the use of imaging equipment.

The sealable surface can be formed of a mesh material, such as a polyester or other polymer mesh, which is coated and/or impregnated with a silicone material. In a certain embodiment, the sealable surface can comprise a warp polymer mesh containing a silicone gel material.

As illustrated in FIG. 5 through FIG. 7, the interspinous process brace can include a superior spinous process pocket 710 and an inferior spinous process pocket 712. Further, a superior spinous process engagement structure 720 can extend from a surface within the superior spinous process pocket 710. Also, an inferior spinous process engagement structure 722 can extend from a surface within the inferior spinous process pocket 710. In a particular embodiment, each of the spinous process engagement structures 720, 722 can be one or more spikes, one or more teeth, a combination thereof, or some other structure configured to engage a spinous process.

FIG. 5 through FIG. 7 indicate that the interspinous process brace 700 can be implanted between a superior spinous process 800 and an inferior spinous process 802. In a particular embodiment, the adjustable component 702 can be inflated so the spinous process pockets 710, 712 engage the spinous processes 800, 802. In a particular embodiment, when the interspinous process brace 700 is properly installed and inflated between the superior spinous process 800 and the inferior spinous process 802, the superior spinous process pocket 710 can engage and support the superior spinous process 800. Further, the inferior spinous process pocket 712 can engage and support an inferior spinous process 802.

More specifically, the superior spinous process engagement structure 720 can extend slightly into and engage the superior spinous process 800. Also, the inferior spinous process engagement structure 722 can extend slightly into and engage the inferior spinous process 802. Accordingly, the spinous process engagement structures 720, 722, the spinous process pockets 710, 712, or a combination thereof can substantially prevent the expandable interspinous process brace 700 from migrating with respect to the spinous processes 800, 802.

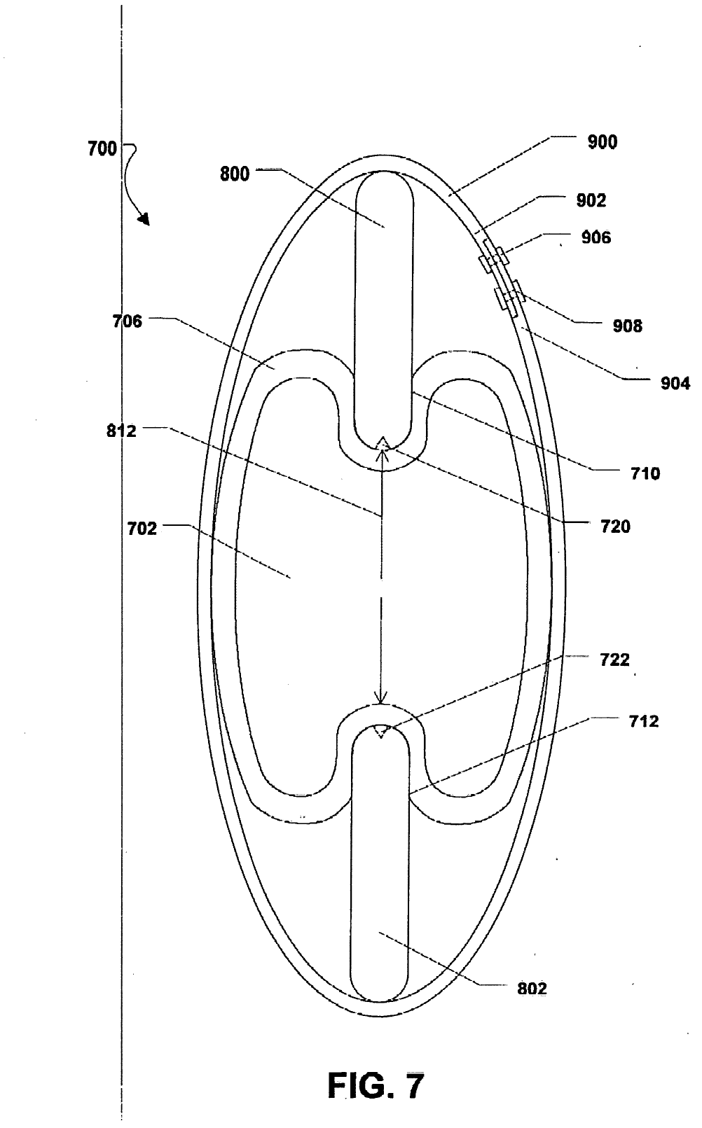

Also, in a particular embodiment, the expandable interspinous process brace can be movable between a deflated configuration, shown in FIG. 5, and one or more inflated configurations, shown in FIG. 6 and FIG. 7. In the deflated configuration, a distance 812 between the superior spinous process pocket 710 and the inferior spinous process pocket 712 can be at a minimum. However, as one or more materials are injected into the adjustable component 702, the distance 812 between the superior spinous process pocket 710 and the inferior spinous process pocket 712 can increase.

Accordingly, the interspinous process brace 700 can be installed between a superior spinous process 800 and an inferior spinous process 802. Further, the interspinous process brace 700 can be expanded, e.g., by injecting one or more materials into the adjustable component 702, in order to increase the distance between the superior spinous process 800 and the inferior spinous process 802 (i.e., to distract the processes).

Alternatively, a distractor can be used to increase the distance between the superior spinous process 800 and the inferior spinous process 802 and the interspinous process brace 700 can be expanded to support the superior spinous process 800 and the inferior spinous process 802. After the interspinous process brace 700 is expanded accordingly, the distractor can be removed and the interspinous process brace 700 can support the superior spinous process 800 and the inferior spinous process 802 to substantially prevent the distance between the superior spinous process 802 and the inferior spinous process 800 from returning to a pre-distraction value.

In a particular embodiment, the interspinous process brace 700 can be initially injected with one or more injectable biocompatible materials. For example, the injectable biocompatible materials can include polymer materials. Also, the injectable biocompatible materials can include ceramics.

For example, the polymer materials can include polyurethanes, polyolefins, silicones, silicone polyurethane copolymers, polymethylmethacrylate (PMMA), epoxies, cyanoacrylates, hydrogels, or a combination thereof. Further, the polyolefin materials can include polypropylenes, polyethylenes, halogenated polyolefins, or fluoropolyolefins.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), polylactic acid (PLA), or a combination thereof.

In a particular embodiment, the ceramics can include calcium phosphate, hydroxyapatite, calcium sulfate, bioactive glass, or a combination thereof. In various embodiments, the ceramics can be provided as beads, powder, microspheres, microrods, or the like. In an alternative embodiment, the injectable biocompatible materials can include one or more fluids such as sterile water, saline, or sterile air.

FIG. 7 indicates that a tether 900 can be installed around the interspinous process brace 700, after the interspinous process brace 700 is initially expanded as described herein. As shown, the tether 900 can include a proximal end 902 and a distal end 904. In a particular embodiment, the tether 900 can circumscribe the interspinous process brace 700 and the spinous processes 800, 802. Further, the ends 902, 904 of the tether 900 can be brought together and one or more fasteners can be installed there through to connect the ends 902, 904. Accordingly, the tether 900 can be installed in order to prevent the distance between the spinous processes 800, 802 from substantially increasing beyond the distance provided by the interspinous process brace 700 after it is expanded and to maintain engagement of the interspinous processes with the spinous process pockets 710, 712, the engagement structures 720, 722, or a combination thereof.

In a particular embodiment, the tether 900 can comprise a biocompatible elastomeric material that flexes during installation and provides a resistance fit against the processes. Further, the tether 900 can comprise a substantially non-resorbable suture or the like.

The interspinous process brace can also include a sensor 707 located partially or fully within the brace, e.g., the adjustable component. Alternatively or in addition, a sensor can be located near the implant site to monitor conditions proximate the brace. The sensor 707 can be configured to be in communication, e.g., electrical contact, with the connector 706 such that information can be relayed from the sensor to a point of use via the connector 706. In a particular embodiment, the connector 706 can include an electrical conductor 708 to communicate a signal from the sensor 707. In various embodiments, the sensor 707 can include a pressure transducer, a moisture sensor, an electrical resistance sensor or any combination thereof.

In use, a performance condition of the implant can be monitored and, if necessary, an agent can be delivered through the connector 706 in order to affect a characteristic of the adjustable component. For example, the monitored condition can be the size of or a pressure within the adjustable component, a hydration level, a pH level, or the like. In response, an agent can be delivered to the adjustable component that affects a characteristic of the adjustable component, such as for example, the size, hardness or rigidity of the adjustable component. In certain embodiments, the degree of crosslinking of the material in the adjustable component can be affected. In certain embodiments, the agent can be delivered to postoperatively customize the implant for fit or use in the recipient.

The delivered agent can generally affect a condition of the spinal implant. More specifically, the agent can affect a condition of the adjustable component of the spinal implant. For example, in the embodiment shown in FIGS. 5-7, the agent can affect a condition of the injected material contained in the adjustable component. For example, the agent can decrease the hydration level of the injected material or can cause a degeneration of the injected material that leads to a reduction in hydration level, to a reduction in pressure, or to a reduction in size of the injected material within the adjustable component. An agent causing degeneration of or reduction in hydration level of the contents of an adjustable component is herein termed a “degrading agent.” In another example, an agent can increase the hydration level of the injected material or can be injected into the adjustable component to increase the size of the adjustable component or in an increase in pressure within the adjustable component. Such an agent that can cause an increase in hydration of or an increase in size of or an increase in pressure in the adjustable component is herein termed a “stimulating agent.” In a further example, an agent (herein termed a “crosslinking agent”) can increase the rigidity, hardness or degree of crosslinking of the material in the adjustable component.

An exemplary degrading agent can reduce hydration levels in the adjustable component, resulting in a reduction in hydration level or in pressure or, when an elastically expandable adjustable component is employed, in volume within the adjustable component. For example, depending on the contents of the adjustable component, the degrading agent can be an art-recognized proteolytic agent that breaks down proteins.

An exemplary stimulating agent can include material identical to that already contained in the adjustable component, which can be injected under pressure to increase the size of, volume of and/or pressure in the adjustable component. Alternatively or in addition, a stimulating agent can include a growth factor. The growth factor can be generally suited to promote the formation of tissues, especially of the type(s) naturally occurring as spinal components. For example, the growth factor can promote the growth or viability of tissue or cell types occurring in the nucleus pulposus, such as nucleus pulposus cells or chondrocytes, as well as space filling cells, such as fibroblasts, or connective tissue cells, such as ligament or tendon cells. Alternatively or in addition, the growth factor can promote the growth or viability of tissue types occurring in the annulus fibrosis, as well as space filling cells, such as fibroblasts, or connective tissue cells, such as ligament or tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

Each of the agents can be maintained and/or introduced in liquid, gel, paste, slurry, semi-solid or solid form, or any combination thereof. Solid forms can include powder, granules, microspheres, miniature rods, or embedded in a matrix or binder material, or any combination thereof. Further, a stabilizer or a preservative can be included with the agent to prolong activity of the agent.

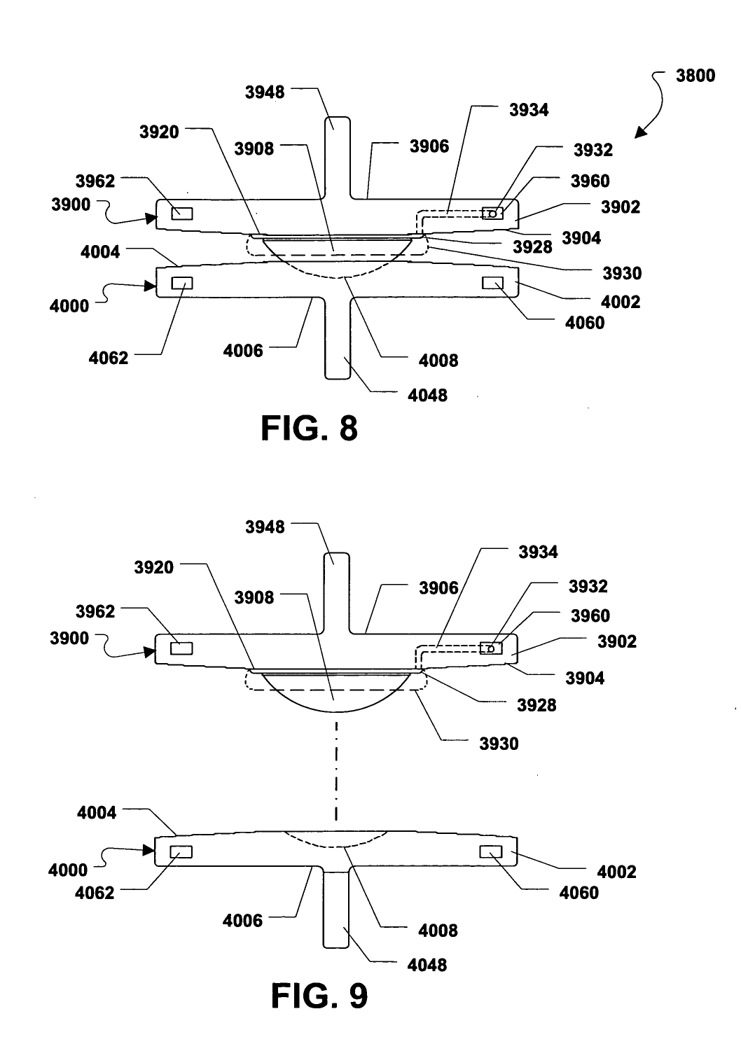

Another aspect of the present disclosure is depicted in FIGS. 8-13, which show an intervertebral prosthetic disc (generally designated 3800). As illustrated, the intervertebral prosthetic disc 3800 can include a superior component 3900 and an inferior component 4000. In a particular embodiment, the components 3900, 4000 can be made from one or more extended use approved medical materials. For example, the materials can be metal containing materials, polymer materials, or composite materials that include metals, polymers, or combinations of metals and polymers.

In a particular embodiment, the metal containing material can be a metal. Further, the metal containing material can be a ceramic. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, fluoropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 3900, 4000 can be made from any other substantially rigid biocompatible materials.

In a particular embodiment, the superior component 3900 can include a superior support plate 3902 that has a superior articular surface 3904 and a superior bearing surface 3906. In a particular embodiment, the superior articular surface 3904 can be generally curved and the superior bearing surface 3906 can be substantially flat. In an alternative embodiment, the superior articular surface 3904 can be substantially flat and at least a portion of the superior bearing surface 3906 can be generally curved.

In a particular embodiment, after installation, the superior bearing surface 3906 can be in direct contact with vertebral bone, e.g., cortical bone and cancellous bone. Further, the superior bearing surface 3906 can be coated with a bone-growth promoting substance, e.g., a hydroxyapatite coating formed of calcium phosphate. Additionally, the superior bearing surface 3906 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 8 through FIG. 13, a projection 3908 can extends from the superior articular surface 3904 of the superior support plate 3902. In a particular embodiment, the projection 3908 can have a hemi-spherical shape. Alternatively, the projection 3908 can have an elliptical shape, a cylindrical shape, or other arcuate shape. Moreover, the projection 3908 can be formed with a groove 3910.

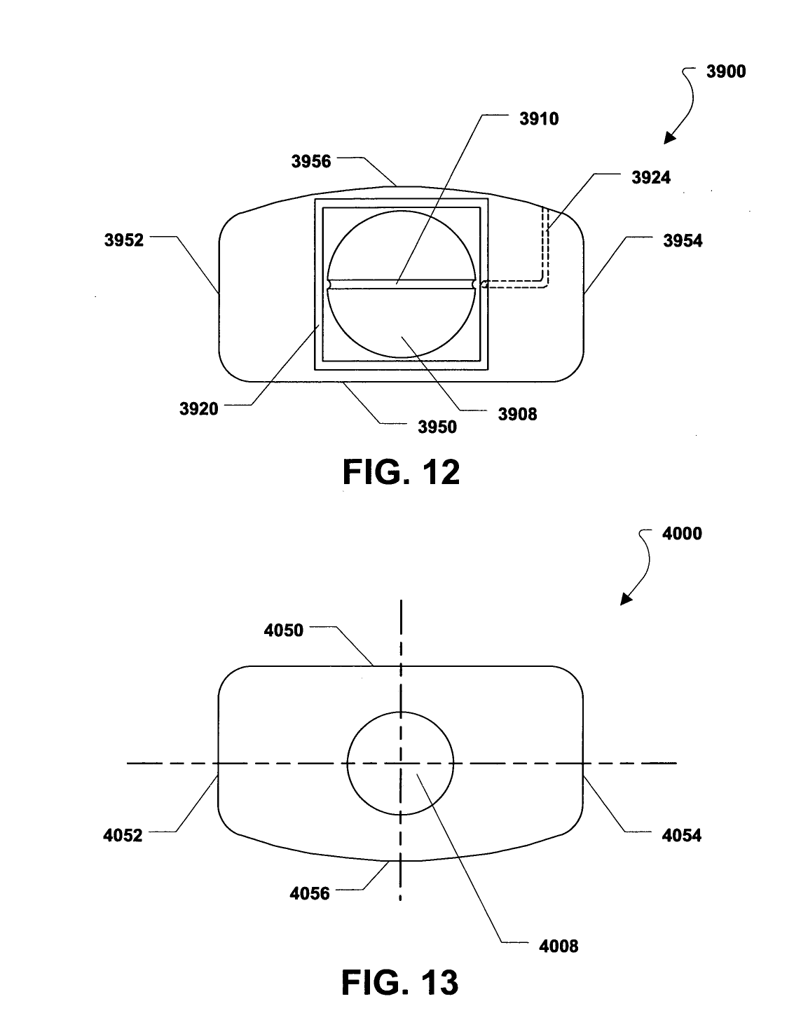

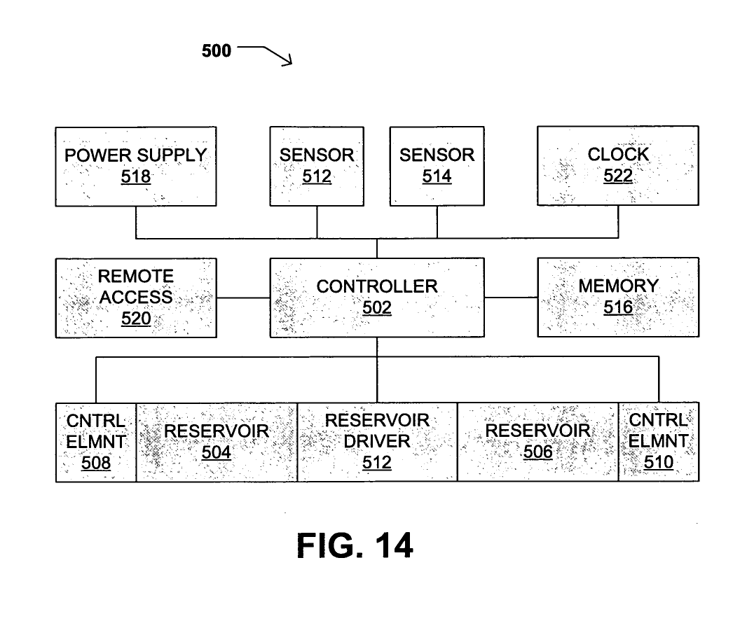

As further illustrated in FIG. 12, the superior component 3900 includes an adjustable component (e.g., an expandable motion limiter) 3920 that is affixed, or otherwise attached to, the superior articular surface 3904. In a particular embodiment, as depicted in FIG. 12, the adjustable component 3920 is generally square and surrounds the projection 3908. Alternatively, the adjustable component 3920 can be generally rectangular, circular or any other polygonal or arcuate shape.

FIG. 8 through FIG. 11 indicate that the adjustable component 3920 can be inflated from a deflated position 3928 to one of a plurality of intermediate inflated positions up to a maximum inflated position 3930. In a particular embodiment, the adjustable component 3920 can be initially injected with one or more injectable biocompatible materials. For example, the injectable biocompatible materials can include polymer materials. Also, the injectable biocompatible materials can include ceramics.

For example, the polymer materials can include polyurethanes, polyolefins, silicones, silicone polyurethane copolymers, polymethylmethacrylate (PMMA), epoxies, cyanoacrylates, hydrogels, or a combination thereof. Further, the polyolefin materials can include polypropylenes, polyethylenes, halogenated polyolefins, or fluoropolyolefins.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), polylactic acid (PLA), or a combination thereof.

In a particular embodiment, the ceramics can include calcium phosphate, hydroxyapatite, calcium sulfate, bioactive glass, or a combination thereof. In various embodiments, the ceramics can be provided as beads, powder, microspheres, microrods, or the like. In an alternative embodiment, the injectable biocompatible materials can include one or more fluids such as sterile water, saline, or sterile air.

In alternative embodiments, the adjustable component can be inflated with one or more of the following: fibroblasts, lipoblasts, chondroblasts, differentiated stem cells or other biologic factor which would create a motion limiting tissue when injected into a bioresorbable motion limiting scaffold.

As shown in FIG. 8 through FIG. 12, the superior support plate 3902 can include a port 3932 that is in fluid communication with a fluid channel 3934 that provides fluid communication to the adjustable component 3920. The adjustable component 3920 can be inflated or adjusted with a material or agent that is delivered to the adjustable component 3920 via the port 3932 and the fluid channel 3934.

The intervertebral prosthetic disc can include a connector (not shown), in communication with the adjustable component 3920, which communication can be accomplished via the fluid channel 3934. The connector can be used to initially provide an injectable biocompatible material to the adjustable component 3920 during installation. Further, after the adjustable component 3920 is initially inflated, or otherwise expanded, the connector can be positioned transcutaneously or attached to a transcutaneous, self-sealable port in order to allow unobstructed, postoperative access to the adjustable component from outside the patient. Alternatively, the connector can include an implantable self-sealing port to allow percutaneous access to the connector.

In another exemplary embodiment, the intervertebral prosthetic disc can include an adjustable component having a sealable surface configured to allow percutaneous delivery of an agent directly to the adjustable component, i.e., without passing through a connector. The sealable surface can be a portion of a side of the implant (e.g., a window), such as a portion of the posterior side. In other embodiments, the sealable surface can comprise the entire side or multiple sides of the implant such that the agent can be delivered percutaneously through a needle with or without the use of imaging equipment. In another exemplary embodiment, the port 3932 that is in fluid communication with the fluid channel 3934 can include a sealable surface that can be accessed percutaneously.

The sealable surface can be formed of a mesh material, such as a polyester or other polymer mesh which is coated and/or impregnated with a silicone material. In a certain embodiment, the sealable surface can comprise a warp polymer mesh containing a silicone gel material.

FIG. 8 through FIG. 11 indicate that the superior component 3900 can include a superior keel 3948 that extends from superior bearing surface 3906. During installation, the superior keel 3948 can at least partially engage a keel groove that can be established within a cortical rim of a vertebra.

As illustrated in FIG. 12, the superior component 3900 can be generally rectangular in shape. For example, the superior component 3900 can have a substantially straight posterior side 3950. A first straight lateral side 3952 and a second substantially straight lateral side 3954 can extend substantially perpendicular from the posterior side 3950 to an anterior side 3956. In a particular embodiment, the anterior side 3956 can curve outward such that the superior component 3900 is wider through the middle than along the lateral sides 3952, 3954. Further, in a particular embodiment, the lateral sides 3952, 3954 are substantially the same length.

FIG. 8 and FIG. 9 show that the superior component 3900 includes a first implant inserter engagement hole 3960 and a second implant inserter engagement hole 3962. In a particular embodiment, the implant inserter engagement holes 3960, 3962 are configured to receive respective dowels, or pins, that extend from an implant inserter (not shown) that can be used to facilitate the proper installation of an intervertebral prosthetic disc, e.g., the intervertebral prosthetic disc 3800 shown in FIG. 8 through FIG. 13.

In a particular embodiment, the inferior component 4000 includes an inferior support plate 4002 that has an inferior articular surface 4004 and an inferior bearing surface 4006. In a particular embodiment, the inferior articular surface 4004 can be generally curved and the inferior bearing surface 4006 can be substantially flat. In an alternative embodiment, the inferior articular surface 4004 can be substantially flat and at least a portion of the inferior bearing surface 4006 can be generally curved.

In a particular embodiment, after installation, the inferior bearing surface 4006 can be in direct contact with vertebral bone, e.g., cortical bone and cancellous bone. Further, the inferior bearing surface 4006 can be coated with a bone-growth promoting substance, e.g., a hydroxyapatite coating formed of calcium phosphate. Additionally, the inferior bearing surface 4006 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 8 through FIG. 11, a depression 4008 can extend into the inferior articular surface 4004 of the inferior support plate 4002. In a particular embodiment, the depression 4008 can be sized and shaped to receive the projection 3908 of the superior component 3900. For example, the depression 4008 can have a hemi-spherical shape. Alternatively, the depression 4008 can have an elliptical shape, a cylindrical shape, or other arcuate shape.

FIG. 8 through FIG. 11 indicate that the inferior component 4000 can include an inferior keel 4048 that extends from inferior bearing surface 4006. During installation, the inferior keel 4048 can at least partially engage a keel groove that can be established within a cortical rim of a vertebra, e.g., the keel groove 410 shown in FIG. 3.

In a particular embodiment, as shown in FIG. 13, the inferior component 4000 can be shaped to match the shape of the superior component 3900, shown in FIG. 12. Further, the inferior component 4000 can be generally rectangular in shape. For example, the inferior component 4000 can have a substantially straight posterior side 4050. A first straight lateral side 4052 and a second substantially straight lateral side 4054 can extend substantially perpendicular from the posterior side 4050 to an anterior side 4056. In a particular embodiment, the anterior side 4056 can curve outward such that the inferior component 4000 is wider through the middle than along the lateral sides 4052, 4054. Further, in a particular embodiment, the lateral sides 4052, 4054 are substantially the same length.

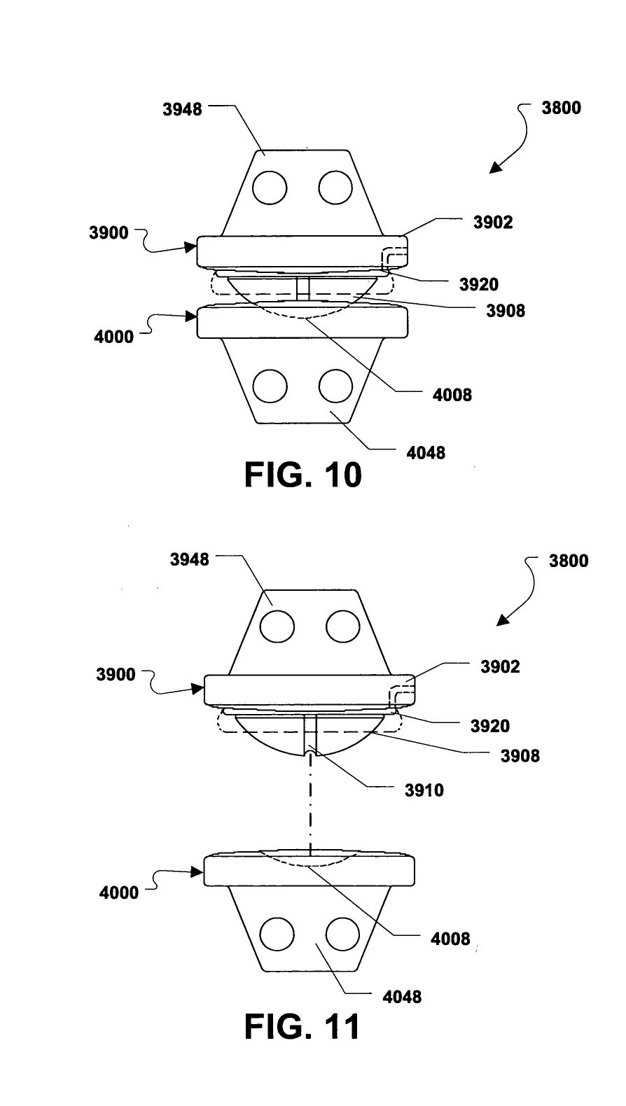

FIG. 8 and FIG. 10 show that the inferior component 4000 includes a first implant inserter engagement hole 4060 and a second implant inserter engagement hole 4062. In a particular embodiment, the implant inserter engagement holes 4060, 4062 are configured to receive respective dowels, or pins, that extend from an implant inserter (not shown) that can be used to facilitate the proper installation of an intervertebral prosthetic disc, e.g., the intervertebral prosthetic disc 3800 shown in FIG. 8 through FIG. 13.

In a particular embodiment, the overall height of the intervertebral prosthetic device 3800 can be in a range from fourteen millimeters to forty-six millimeters (14-46 mm). Further, the installed height of the intervertebral prosthetic device 3800 can be in a range from eight millimeters to sixteen millimeters (8-16 mm). In a particular embodiment, the installed height can be substantially equivalent to the distance between an inferior vertebra and a superior vertebra when the intervertebral prosthetic device 3800 is installed there between.

In a particular embodiment, the length of the intervertebral prosthetic device 3800, e.g., along a longitudinal axis, can be in a range from thirty millimeters to forty millimeters (30-40 mm). Additionally, the width of the intervertebral prosthetic device 3800, e.g., along a lateral axis, can be in a range from twenty-five millimeters to forty millimeters (25-40 mm). Moreover, in a particular embodiment, each keel 3948, 4048 can have a height in a range from three millimeters to fifteen millimeters (3-15 mm).

Although depicted in the Figures as a two piece-design, in alternative embodiments, multiple-piece designs can be employed. For example, in an alternative embodiment, the projection 3908 is not fixed or unitary with either of the support plates 3902, 4002 and, instead, is configured as a substantially rigid spherical member (not shown) that can independently articulate with each support plate 3902, 4002. Additionally or alternatively, each component can comprise multiple components (not shown). These components can articulate with or be fixed to the support plates 3902, 4002. Furthermore, adjustable components can be configured to limit relative motion between any of the components described above or among multiple components.

The intervertebral prosthetic disc can also include a sensor (not shown) located partially or fully within the disc, e.g., in the adjustable component. Alternatively or in addition, a sensor can be located near the implant site to monitor conditions proximate the disc. The sensor can be configured to be in communication, e.g., electrical contact, with the connector such that information can be relayed from the sensor to a point of use via the connector. In a particular embodiment, the connector can include an electrical conductor to communicate a signal from the sensor. In various embodiments, the sensor can include a pressure transducer, a moisture sensor, an electrical resistance sensor or any combination thereof.

In use, a performance condition of the implant can be monitored and, if necessary, an agent can be delivered through the connector 706 in order to affect a characteristic of the adjustable component. For example, the monitored condition can be the size of or a pressure within the adjustable component, a hydration level, a pH level, or the like. Further, the patient can be manually monitored for pain, range of motion, or the like. In response, an agent can be delivered to the adjustable component that affects a characteristic of the adjustable component, such as for example, the size, hardness or rigidity of the adjustable component. In certain embodiments, the degree of crosslinking of the material in the adjustable component can be affected. In certain embodiments, the agent can be delivered to postoperatively customize the implant for fit or use in the recipient.

The delivered agent can generally affect a condition of the spinal implant. More specifically, the agent can affect a condition of the adjustable component of the spinal implant. For example, in the embodiment shown in FIGS. 8-13, the agent can affect a condition of the injected material contained in the adjustable component. For example, the agent can decrease the hydration level of the injected material or can cause a degeneration of the injected material that leads to a reduction in hydration level, to a reduction in pressure, or to a reduction in size of the injected material within the adjustable component. An agent causing degeneration of or reduction in hydration level of the contents of an adjustable component is herein termed a “degrading agent.” In another example, an agent can increase the hydration level of the injected material or can be injected into the adjustable component to increase the size of the adjustable component or in an increase in pressure within the adjustable component. Such an agent that can cause an increase in hydration of or an increase in size of or an increase in pressure in the adjustable component is herein termed a “stimulating agent.” In a further example, an agent (herein termed a “crosslinking agent”) can increase the rigidity, hardness or degree of crosslinking of the material in the adjustable component.

An exemplary degrading agent can reduce hydration levels in the adjustable component, resulting in a reduction in hydration level or in pressure or, when an elastically expandable adjustable component is employed, in volume within the adjustable component. For example, depending on the contents of the adjustable component, the degrading agent can be an art-recognized proteolytic agent that breaks down proteins.

An exemplary stimulating agent can include material identical to that already contained in the adjustable component, which can be injected under pressure to increase the size of, volume of and/or pressure in the adjustable component. Alternatively or in addition, a stimulating agent can include a growth factor. The growth factor can be generally suited to promote the formation of tissues, especially of the type(s) naturally occurring as spinal components. For example, the growth factor can promote the growth or viability of tissue or cell types occurring in the nucleus pulposus, such as nucleus pulposus cells or chondrocytes, as well as space filling cells, such as fibroblasts, or connective tissue cells, such as ligament or tendon cells. Alternatively or in addition, the growth factor can promote the growth or viability of tissue types occurring in the annulus fibrosis, as well as space filling cells, such as fibroblasts, or connective tissue cells, such as ligament or tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

Each of the agents can be maintained and/or introduced in liquid, gel, paste, slurry, semi-solid or solid form, or any combination thereof. Solid forms can include powder, granules, microspheres, miniature rods, or embedded in a matrix or binder material, or any combination thereof. Further, a stabilizer or a preservative can be included with the agent to prolong activity of the agent.

In addition to the interspinous process brace and intervertebral prosthetic disc embodiments shown in the present figures, the general configuration disclosed herein can be utilized with other implants, such as partial or full nucleus replacement implants. In such embodiments, the nucleus replacement can include an adjustable component comprising an expandable or otherwise fillable compartment that is disposed in an intervertebral disc, such as within the annulus fibrosis. The adjustable component can be initially filled during installation and, thereafter, adjusted, configured or customized by delivering an agent to the adjustable component through a connector—as described previously.

In addition to a design that provides for external access to an adjustable component of a spinal implant, an additional aspect of the present disclosure is directed to an implant control device that can provide multiple adjustments to an implant based on a performance condition or other criterion(ia). In a particular embodiment, an implant control device includes a sensor, a controller, and a reservoir to store an agent. FIG. 6 includes an illustration of an exemplary device 500. The exemplary device 500 includes a controller 502. At least one sensor 512, 514, such as the sensors described above in connection with an interspinous process brace and an intervertebral prosthetic disc, can be in communication with the controller 502. The sensors can be configured to determine a performance condition associated with a spinal implant, such as any of the implants described herein. In addition, the device 500 can include a reservoir, such as the reservoirs 504 and 506. The controller 502 can be communicatively coupled to a control element, such as the control elements 508 and 510, associated with the reservoir, such as the reservoirs 504 and 506, respectively. In addition, the controller 502 can be communicatively coupled to a reservoir driver 512 that can motivate movement of an agent from the reservoir, such as the reservoirs 504 and 506.

In an exemplary embodiment, the controller 502 can receive a signal from the sensor and in response, manipulate the control element 508 or 510. For example, the controller 502 can include control circuitry, such as an algorithmic or arithmetic control circuitry. In an example, the controller 502 includes a proportional, integral, or differential (PID) controller. Alternatively, the controller 502 can include a processor configured to received sensor data, such as data from the sensor, and determine a dosage to be delivered. Based on the dosage, the processor can manipulate the control elements 508 or 510 or the reservoir driver 512. For example, the controller 502 can apply sensor data to an algorithm, an arithmetic model, an artificial intelligence engine, a threshold, or any combination thereof to determine a dosage or control protocol. An exemplary artificial intelligence engine includes a neural network, a fuzzy logic engine, a complex control model, or any combination thereof. In a further example, the controller 502 can perform calculations using the sensor data to determine, for example, a time average, a minimum value, a maximum value, a median value, a rate of change, a trend, or any combination thereof. Further, measurements can be selected or selectively weighted based on the time of day in which taken. For example, pressure data measured at a time at which a patient is typically asleep can be selected in contrast to pressure data measured during periods of high activity.

In an exemplary embodiment, the device 500 includes one or more sensors. An exemplary sensor can include a pressure transducer, a moisture or hydration sensor, a pH sensor, a resistance or conductance meter, an electrolyte detector, or any combination thereof. Based on signals produced by the one or more sensors (512 or 514), the controller 502 can selectively initiate the release of an agent. In addition, the controller 502 can store sensor data in a memory 516.

The device 500 can also include one or more reservoirs, such as reservoirs 504 or 506. The reservoir (504 or 506) can include an agent, such as a stimulating agent or a degrading agent or a crosslinking agent (as previously described). In a particular example, the device 500 includes a reservoir 504 that includes a stimulating agent and includes a reservoir 506 that includes a degrading agent. The reservoirs (504 or 506) can be configured to store the agent in a liquid, gel, paste, slurry, or solid forms, or any combination thereof. A solid form can include powder, granule, microsphere, miniature rod, agent embedded in a matrix or binder material, or any combination thereof. In a solid form example, fluids or water from surrounding tissues can be absorbed by the device 500 and placed in contact with an agent in solid form prior to release. In a further example, the reservoir (504 or 506) can include a refill port, such as a percutaneous refill port.

A reservoir driver 512 can be coupled to the reservoir (504 or 506). As illustrated, the reservoir driver 512 can be coupled to both the reservoir 504 and the reservoir 506. Alternatively, a separate reservoir driver can be connected to each reservoir (504 or 506). An exemplary reservoir driver 512 can include a pump. For example, a pump can add fluid or water from surrounding tissue to a chamber that applies pressure to the reservoir (504 or 506), motivating an agent from the reservoir (504 or 506). In another example, the pump can add water or fluid directly to the reservoir (504 or 506) to increase pressure within the chamber or to hydrate a solid form agent within the reservoir (504 or 506).

In another example, the reservoir driver 512 can include an osmotic driver. For example, a membrane can separate a chamber from surrounding tissue. An osmotic agent within the chamber can absorb water or fluid from the surrounding tissue and expand or increase pressure within the chamber. The osmotic agent can include a non-volatile water-soluble osmagent, an osmopolymer that swells on contact with water, or a mixture of the two. An osmotic agent, such as sodium chloride with appropriate lubricants, binders, or viscosity modifying agents, such as sodium carboxymethylcellulose or sodium polyacrylate can be prepared in various forms. Sodium chloride in tablet form is a water swellable agent. The osmotic agent can generate between about 0 and about 36 MPa (about 5200 psi) of pressure. Materials suitable for the fluid permeable membrane include those that are semipermeable and that can conform to the shape of the housing upon wetting and make a watertight seal with the rigid surface of the housing. The polymeric materials from which the membrane can be made vary based on the pumping rates and device configuration requirements and can include plasticized cellulosic materials, enhanced polymethylmethacrylate such as hydroxyethylmethacrylate (HEMA), elastomeric materials such as polyurethanes and polyamides, polyether-polyamide copolymers, thermoplastic copolyesters, or the like, or any combination thereof. The chamber can apply pressure to a movable barrier between the chamber and the reservoir (504 or 506), motivating agent from the reservoir (504 or 506).

In a further example, the reservoir driver 512 can include a mechanical system that motivates agent from the reservoir (504 or 506). For example, the mechanical system can include a piston, a rotating screw, or any combination thereof.

In the exemplary device 500, a control element, such as the control elements 508 or 510, can be connected to the reservoir, such as the reservoirs 504 or 506, respectively. The control element (508 or 510) can permit access to the respective reservoir (504 or 506). For example, the control element (508 or 510) can include a valve that permits fluid agent to exit the reservoir (504 or 506). In another example, the control element (508 or 510) can include a pump that removes fluid agent from the reservoir (504 or 506). In a further example, the control element (508 or 510) can include a door that permits solid form agent to be pushed from the reservoir (504 or 506).

In an exemplary embodiment, the control element (508 or 510) and the reservoir driver 512 can be the same device. For example, a pump can both motivate the agent from the reservoir (504 or 506) and control the flow of the agent. In another example, a mechanical driver can act to both motivate and control the amount of agent exiting the reservoir (504 or 506).

In a further exemplary embodiment, the control element (508 or 510) can include a destructible or removable barrier. For example, individual reservoirs (504 or 506) can include a single dose of an agent. An array of reservoirs can be provided that each includes a removable barrier. Destruction or removal of the barrier exposes the contents of the reservoir to surrounding tissue. For example, the barrier can be a thin film that bursts when an agent within the reservoir is heated or activated. In another example, the barrier can be a film that when heated or exposed to electric current disintegrates, exposing a reservoir.

The device 500 can also include a memory 516 in communication with the controller 502. The controller 502 can store sensor data at the memory 516. In another example, the controller 502 can store parameter values that are accessed to determine control actions. For example, the controller 502 can store threshold values, model parameters, dosage parameters, or any combination thereof at the memory 516. As illustrated, the controller 502 is directly coupled to the memory 516. Alternatively, the controller 502 can communicate with a memory controller that in turn controls the memory 516. An exemplary memory 516 can include random access memory (RAM).

In addition, the device 500 can include a clock 522. The clock 522 can provide a time signal to the controller 502. The controller 502, for example, can use the time signal to time stamp sensor data. In another example, the controller 502 can use the time signal in performing calculations based on the sensor signal. For example, the controller 502 can select or weight sensor signals based on time of day. In another example, the controller can determine a minimum or maximum value of the sensor signal for a 24-hour period. In a further example, the controller 502 can determine a rate of change or a trend based on the time signal and sensor data.

The device 500 can further include a power supply 518. For example, the power supply 518 can include a battery. In an exemplary embodiment, the battery is a rechargeable battery. The power supply 518 can include a wireless power regeneration circuitry, such as an induction coil, or can include a recharging port. For example, the induction coil can respond to an electromagnetic signal and generate power for storage in a battery. In the example illustrated, the power supply 518 is coupled to the controller 502.

In an exemplary embodiment, the device 500 can include a remote access component 520. The remote access component 520 can be in communication with the controller 502. In an example, the remote access component 520 can respond to a magnetic field. In another example, the remote access component 520 can respond to an electromagnetic signal, such as a radio frequency signal. In a further example, the remote access component 520 can respond to a light signal, such as an infrared signal. In an additional example, the remote access component 520 can respond to a wave signal, such as an ultrasonic signal.

In response to a signal from the remote access component 520, the controller 502 can activate or change mode. In an example, the controller 502 can initiate control of the control element (508 or 510) or reading of the sensor (512 or 514) in response to a signal from the remote access component 520. In another example, the controller 502 can cease control or reading of components in response to a signal from the remote access component 520. In another exemplary embodiment, the controller 502 can communicate data via an antenna included within the remote access component 520. For example, sensor data stored in the memory 516 can be transmitted via the antenna.

In a further exemplary embodiment, the remote access component 520 can receive data for use by the controller 502. For example, the data can include control parameters, dosage parameters, timing parameters for data storage, time and date, programming instructions, or any combination thereof. An exemplary control parameter includes a threshold value, an algebraic constant, a model parameter, or any combination thereof.

In an alternative embodiment, the device can include a remote access component 520 that directly manipulates the control element (508 or 510) or the reservoir driver 512. For example, the remote access component 520 can directly manipulate the control element 508, such as a valve. In another example, the remote access component 520 can directly manipulate the reservoir driver 512. In a particular example, the device 500 includes a reservoir 504 including an agent, a reservoir driver 512 coupled to the reservoir and configured to effect the release of the agent from the reservoir 504, and a remote access component 520. In this particular example, the device 500 can be configured to manipulate the reservoir driver 512 to effect the release of the agent in response to a first signal received via the remote access component 520. For example, the control element 508 can be a valve that opens or closes in response to pressure in the reservoir 504. The reservoir driver 512 can increase the pressure in the reservoir 504 to open or close the valve. In addition, the device 500 can be configured to manipulate the reservoir driver 512 to prevent release of the agent in response to a second signal received via the remote access component 520.

In a further example, the device 500 can include a second reservoir 508 including a second agent. For example, the first agent can be a degrading agent and the second agent can be a stimulating agent. In a device including a single reservoir driver 508, the reservoir driver 512 can be coupled to the second reservoir 508. In another embodiment, the device 500 can include a second reservoir driver coupled to the second reservoir 508. The device 500 can be configured to manipulate the second reservoir driver to effect the release of the second agent. In a particular embodiment, the remote access component 520 can be configured to communicate using an IEEE 802.15 communication protocol.

In a particular example, a patient in which the device 500 is implanted can experience pain or a test of the patient, such as a computed tomography (CT) scan or a magnetic resonance imaging (MRI) scan, can indicate a problem with the associated spinal implant. A healthcare provider can manipulate the performance of the device 500 by accessing the remote access component 520.

The device, such as device 500 illustrated in FIG. 5, can be included in a housing. The housing can form a cylinder, sphere, capsule, disc, cone, coil shape, or any combination thereof. In an example, the housing can surround each of the components of the device. Alternatively, the individual components can be included within one or more housings. For example, controller can be included in a housing. The reservoir can be at least partially included within the housing, can extend beyond the boundaries of the housing, or can be separate from the housing. In another example, the sensor can be included in a housing with the controller, and the power supply and the remote access component can be housed separately.

The housing can have a largest dimension not greater than about 8 mm. For example, the largest dimension can be not greater than about 5 mm, such as not greater than about 3 mm. In a particular example, a cylindrical housing can have a diameter that is not greater than about 8 mm. In an exemplary capsule-shaped housing, the diameter around the center is not greater than about 8 mm.

The housing can be formed of a metallic material, a polymeric material, or any combination thereof. An exemplary polymeric material can include polypropylene, polyethylene, halogenated polyolefin, fluoropolyolefin, polybutadiene, polysulfone, polyaryletherketone, polyurethane, polyester, or copolymers thereof, silicone, polyimide, polyamide, polyetherimide, a hydrogel, or any combination thereof. An exemplary polyaryletherketone (PAEK) material can include polyetherketone (PEK), polyetheretherketone (PEEK), polyetherketoneketone (PEKK), polyetherketoneetherketoneketone (PEKEKK), or any combination thereof. An exemplary silicone can include dialkyl silicones, fluorosilicones, or any combination thereof. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof. An exemplary metallic material includes stainless steel, titanium, platinum, tantalum, gold or their alloys as well as gold-plated ferrous alloys, platinum-plated ferrous alloys, cobalt-chromium alloys or titanium nitride coated stainless steel, or any combination thereof.

Another aspect is directed to a method of treating a spine. The method can include determining a post surgical performance condition associated with a previously installed spinal implant such as, for example, one of the spinal implants described previously herein. The method can include the step of selectively releasing an agent to affect the performance condition of the previously installed implant, such as by affecting a characteristic of the implant, such as by affecting a characteristic of an adjustable component of the implant. The agent can be delivered transcutaneously via a transcutaneous connector via a syringe or other delivery device. Alternatively, the agent can be delivered from an implanted control device which can itself monitor or provide for external monitoring of a performance condition. In certain embodiments, the step of determining the post surgical performance condition can also be performed using a transcutaneous connector, such as an electrical connector in communication with a sensor proximate or within the implant.

In an exemplary method, an implant control device can be employed. The device can include a controller that measures a condition of a previously installed spinal implant and can release an agent based on the measurement. The implant control device can determine a condition associated with the previously installed spinal implant. The device can be itself implantable or can be placed in transcutaneous communication with a sensor. For example, the sensor can include a pressure sensor, moisture sensor, resistivity or conductivity sensor, pH sensor, or any combination thereof. The device can use signals from the one or more sensors to determine a condition of the implant. For example, a high average pressure measurement or a pressure measurement that is too high at a particular time of day can indicate excess hydration in the adjustable component. In contrast, a low average pressure measurement can indicate a low hydration. In another example, the moisture sensor can indicate a high or low hydration level. In a further example, a combination of pressure data and moisture data can be used in determining the condition of the implant. In an additional example, a trend in data from one or more sensor or a rate of change of a sensor measurement can be used in determining the condition of the implant.

Based on the condition of the implant, the controller can determine a control strategy. For example, the controller can select an agent to be dispensed and can determine a dosage to be dispensed. In a particular example, the controller can release agents in accordance with the control strategy. For low pressure or hydration levels, a stimulating agent can be released. For a moderate pressure or hydration level, no agent is released, and for a high pressure or hydration level, a degrading agent can be released. In certain embodiments, the information regarding the condition can be processed by a technician, who can administer an appropriate agent through a transcutaneous connector.

In response to determining the condition of the implant, the controller can initiate the release of an agent. For example, the controller can selectively release an agent from a reservoir based on the condition. In a particular example, the controller can select an agent to release, determine a dosage or amount of agent to release, and manipulate a control element, based on the determined condition of the implant.

In a particular embodiment, the device can access pressure data. For example, the device can receive pressure data from a sensor or can retrieve pressure data from memory. The device can average the pressure data, such as determine a time average mean of the pressure data. In another example, the device can average a minimum pressure or a maximum pressure for a set of days. In a further example, the device can average pressure measured at a particular time of day, such as when a patient is inactive.

The device can compare the average of the pressure data to a threshold. For example, the threshold can be a low level threshold below which a stimulating agent is to be released. In another example, the threshold can be a high level threshold above which a degrading agent is to be released.

Based on the comparison to the threshold, the device can release an agent. For example, a controller can activate a control element associated with a reservoir including the agent to be released. In another example, the controller can activate a reservoir driver. In certain embodiments, the information regarding the condition can be compared to the threshold by a technician, who can administer an appropriate agent through a transcutaneous connector.

In another exemplary embodiment, a model can be used to determine when and how much agent is to be released. For example, data can be measured by one or more sensors. The data can be applied to a model to determine a condition of the implant or determine dosages and agents to be release in association with the condition of the implant. An exemplary model can include an algebraic model, a neural network model, a fuzzy logic model, or any combination thereof.

Based on the output of the model, the device can initiate release of a first or a second agent. In certain embodiments, the data regarding the condition can be applied to a model by a technician, who can administer an appropriate agent through a transcutaneous connector.

In certain embodiments, the implant control device can itself be implanted and can include an access port to transfer data, such as dosage data and control data into the device. In another example, the device can include a wireless access circuitry, such as a radiofrequency circuitry, an infrared circuitry, or an ultrasonic circuitry for receiving data. In an example, the wireless access circuitry can be proprietary or can conform to a wireless communication standard, such as IEEE 802.11, IEEE 802.15, or IEEE 802.16. In a particular example, the wireless access circuitry is Bluetooth® compatible. Software can be provided to configure the device for a particular patient.

A remote access device located external to the patient can communicate with the remote access component of the device. For example, the remote access device can read data from the device. In another example, the remote access device can transmit parameters or programming instructions to the device. In a particular embodiment, the remote access device can be connected to a computer via a wired connection or a wireless connection.

In an alternative embodiment, the remote access device can be located at a patient's home. A patient can use the remote access device to collect data from the implanted device and forward the data to a physician via the Internet. In addition, the patient can enter additional information via the remote access device or a computer, such as observations and information about painful events. In a particular example, the remote device can connect over a wired or wireless Internet connection to transmit data to a healthcare practitioner and to receive instructions and parameters from the healthcare practitioner. The remote device can connect directly. Alternatively, the remote device can connect to a computer connected to the Internet. In either case, the remote device can access software, either embedded or at a connected computer, to permit entry of comments by the patient in addition to data received from the implanted device. Furthermore, the computer connected to the device or the device itself can provide instructions to the patient. In such a manner, a remotely located healthcare practitioner can remotely monitor performance of the device, the condition of the patient, and manipulate performance of the device.

In a particular example, data retrieved from the implanted device via the remote device can be correlated with pain or sensations experienced by the patient. Such a correlation can further enhance the understanding of the healthcare provider, potentially enhancing the treatment of the patient.

It will be understood that each of the elements described above, or two or more together, may also find utility in applications differing from the types described herein. While the subject matter has been illustrated and described as embodied in an in vivo customizable implant, it is not intended to be limited to the details shown, since various modifications and substitutions can be made without departing in any way from the spirit of the present disclosure. For example, although many examples of various alternative biocompatible chemicals and materials have been presented throughout this specification, the omission of a possible item is not intended to specifically exclude its use in or in connection with the claimed invention. As such, further modifications and equivalents of the subject matter herein disclosed may occur to persons skilled in the art using no more than routine experimentation, and all such modifications and equivalents are believed to be within the spirit and scope of the invention as defined by the following claims.

1. A spinal implant, comprising:
an adjustable component and
a connector in communication with the adjustable component, wherein the connector is configured for transcutaneous delivery of an agent to the adjustable component in a manner that affects a condition of the adjustable component.

2. The spinal implant of claim 1, wherein the adjustable component comprises an expandable component.

3. The spinal implant of claim 1, wherein the connector comprises a catheter having a lumen there through.

4. The spinal implant of claim 1, wherein the connector has a proximal end proximate the adjustable component and a distal end opposite the proximal end.

5. The spinal implant of claim 4, wherein the connector is sealable at the distal end.

6. The spinal implant of claim 1, further comprising a sensor.

7. The spinal implant of claim 6, wherein the connector further comprises an electrical conductor.

8. The spinal implant of claim 7, wherein the sensor is in communication with the electrical conductor.

9. The spinal implant of claim 6, wherein the sensor is disposed at least partially within the spinal implant.

10. The spinal implant of claim 6, wherein the sensor includes a pressure transducer.

11. The spinal implant of claim 6, wherein the sensor includes a moisture sensor.

12. The spinal implant of claim 6, wherein the sensor includes an electrical resistance sensor.

13. The spinal implant of claim 6, further comprising a transmitter in communication with the sensor and configured to relay information concerning the performance condition to a remote location.

14. The spinal implant of claim 1, wherein the condition affected is the size of the adjustable component.

15. The spinal implant of claim 1, wherein the condition affected is the hardness of the adjustable component.

16. The spinal implant of claim 1, wherein the condition affected is the rigidity of the adjustable component.

17. The spinal implant of claim 1, wherein the adjustable component comprises a polymer and the condition affected is the degree of crosslinking of the polymer.

18. The spinal implant of claim 1, wherein the spinal implant is an interspinous process brace.

19. The spinal implant of claim 1, wherein the spinal implant is an intervertebral disc prosthesis.

20. The spinal implant of claim 19, wherein the adjustable component is a motion limiting projection.

21. The spinal implant of claim 1, wherein the spinal implant is a nucleus implant.

22. The spinal implant of claim 4, further comprising an external controller in communication with the distal end of the connector.

23. The spinal implant of claim 4, wherein the distal end of the connector is configured to be removably attachable to a reservoir containing the agent to be delivered to the adjustable component.

24. A spinal implant, comprising:
an adjustable component and
a connector in communication with the adjustable component, wherein the connector comprises an implantable self-sealing port and is configured for percutaneous delivery of an agent to the adjustable component in a manner that affects a condition of the adjustable component.

25. A spinal implant, comprising an adjustable component having a self-sealing surface configured to allow percutaneous delivery of an agent to the adjustable component in a manner that affects a condition of the adjustable component.

26. The spinal implant of claim 25, wherein the self-sealing surface comprises a mesh material.

27. The spinal implant of claim 26, wherein the self-sealing surface further comprises a silicone material.

28. The spinal implant of claim 27, wherein the mesh material comprises a polyester.

29-149. (canceled)
